(19) United States
(12) Patent Application Publication
(10) Pub. No.: US 2009/0194102 A1
(43) Pub. Date: Aug. 6, 2009

(54) Clip Device and Laryngeal Mask Airway

(76) Inventors: Tien-Sheng Chen, Taipei City (TW); Hui-Bih Yuan, Taipei City (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/342,191

(22) Filed: Dec. 23, 2008

(30) Foreign Application Priority Data
Jan. 31, 2008 (TW) 097103785

(51) Int. Cl. A61M 16/06

(52) U.S. Cl. 128/202.13; 128/207.15

(57) ABSTRACT

The clip device of the invention is used for clipping a laryngeal mask airway. The laryngeal mask airway comprises a gas filled portion and a tube portion. The clip device comprises a main portion and a handle portion, wherein the main portion can connect to the laryngeal mask airway. The handle portion is connected to the main portion, and the handle portion is long and thin in shape.

### BACKGROUND OF THE INVENTION

1. Field of the Invention

The present invention relates to a medical instrument; more particularly, the present invention relates to a clip device for clipping a laryngeal mask airway.

2. Description of the Related Art

In the case of anaesthesia or apnea patients, the top priority is the provision of pulmonary ventilation by emergency medical personnel. Among the various respiration sustaining instruments, the laryngeal mask airway (LMA), due to its ease of operability, is a common alternative to endotracheal intubation.

Please refer to FIG. 1 for the structure of a conventional LMA 10P; basically, the LMA 10P mainly comprises a flexible tubular portion 11P, an inflatable sealing cuff 13P, and an inflation tube 15P, wherein the flexible tubular portion 11P comprises a first opening part 111P and a second opening part 112P, the inflatable sealing cuff 13P is disposed surrounding the first opening part 111P, and the inflation tube 15P has one end connected to the inflatable sealing cuff 15P and the other end directing gas into the inflatable sealing cuff 13P.

Please refer to FIGS. 2 to 4 for illustrative diagrams showing the conventional laryngeal mask 10P in use. First, a user may insert the LMA 10P into a patient's mouth by the end having the inflatable sealing cuff 13P, which is in a deflated condition. When the inflatable sealing cuff 13P reaches a deeper part of the mouth, the user will then have to insert his/her finger into the patient's mouth to bend the front end of the LMA 10P to make the structure of the LMA 10P conform with the structure of the patient's upper jaw. In addition, due to the softness of the LMA 10P, the user may not exert force thereby; thus, the user has to push aside the tongue with his/her finger to bend the first opening part 111P and the flexible tubular portion 11P to such an extent that they can reach past the upper jaw to the opening of the trachea, as shown in FIG. 3. In FIG. 4, it is shown that when the LMA 10P gets to a specific position, the user may then aerate the inflatable sealing cuff 13P from the inflation tube 15P so as to form a sealing mask in the patient's throat. The sealing mask may encompass the opening of the trachea and form an air passage thereby; after that, the user may direct gas, such as oxygen, from the second opening part 112P to maintain the patient's respiration.

Accordingly, during the installation of an LMA, users always have to insert their finger(s) (especially their index fingers) into a patient's mouth to pass the soft, inconvenient LMA through the upper jaw because of the obstruction caused by the patient's tongue. In general, this displacement of fingers may cause two problems. First, there is the risk that the user may be bitten by the patient during the installation of an LMA. Second, in a case where a patient's oral space is overly small, which may be caused by an overly small mouth, an overly tight jaw joint, or an overly thick tongue, a user may encounter difficulty inserting his/her finger(s) into the patient's mouth, resulting in failure to install the LMA.

Therefore, it is desirable to provide a clip device for clipping a laryngeal mask airway to mitigate and/or obviate the aforementioned problems.

### SUMMARY OF THE INVENTION

It is an object of the present invention to provide a clip device for clipping a laryngeal mask airway.

It is another object of the present invention to provide a laryngeal mask airway for connecting to the clip device.

To achieve the aforementioned objects, the clip device of the present invention comprises a main portion and a handle portion. The main portion connects to the laryngeal mask airway (LMA); the handle portion is connected to the main portion, and the handle portion is long and thin in shape.

To achieve another aforementioned object, the LMA of the present invention comprises a tube portion, a gas filled portion and at least one protection cover. The tube portion comprises a first end and a second end; the gas filled portion is connected to the first end of the tube portion, and the gas filled portion comprises an upper side and a lower side; and the at least one protection cover is connected to the gas filled portion, and the at least one protection cover is used for holding at least part of the main portion.

According to one of the preferred embodiments of the present invention, the main portion is a C-shaped frame, and the main portion can clip the tube portion of the LMA.

According to another embodiment of the present invention, the number of the at least one protection cover is two, and each of the protection covers respectively covers at least part of the upper side and at least part of the lower side.

Other objects, advantages, and novel features of the invention will become more apparent from the following detailed description when taken in conjunction with the accompanying drawings.

### BRIEF DESCRIPTION OF THE DRAWINGS

These and other objects and advantages of the present invention will become apparent from the following description of the accompanying drawings, which disclose several embodiments of the present invention. It is to be understood that the drawings are to be used for purposes of illustration only, and not as a definition of the invention.

In the drawings, wherein similar reference numerals denote similar elements throughout the several views:

FIGS. 1 to 4 are illustrations of a laryngeal mask airway (LMA) used in the prior art.

FIG. 5 is an illustration of a first embodiment of a clip device of the present invention.

FIG. 6 is an illustration of the first embodiment of the clip device installed on an LMA of the present invention.

FIG. 7 is an illustration of a second embodiment of the clip device of the present invention.

FIG. 8 is an illustration of the second embodiment of the clip device installed on the LMA of the present invention.

FIG. 9 is an illustration of a third embodiment of the clip device of the present invention.

FIG. 10 is an illustration of the third embodiment of the clip device installed on the LMA of the present invention.

FIG. 11 is an illustration of a fourth embodiment of the clip device installed on the LMA of the present invention.

FIG. 12 is an illustration of a fifth embodiment of the clip device installed on the LMA of the present invention.

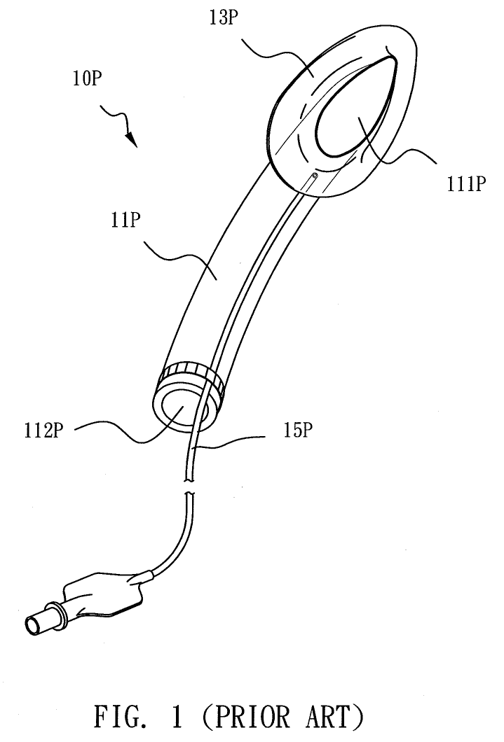

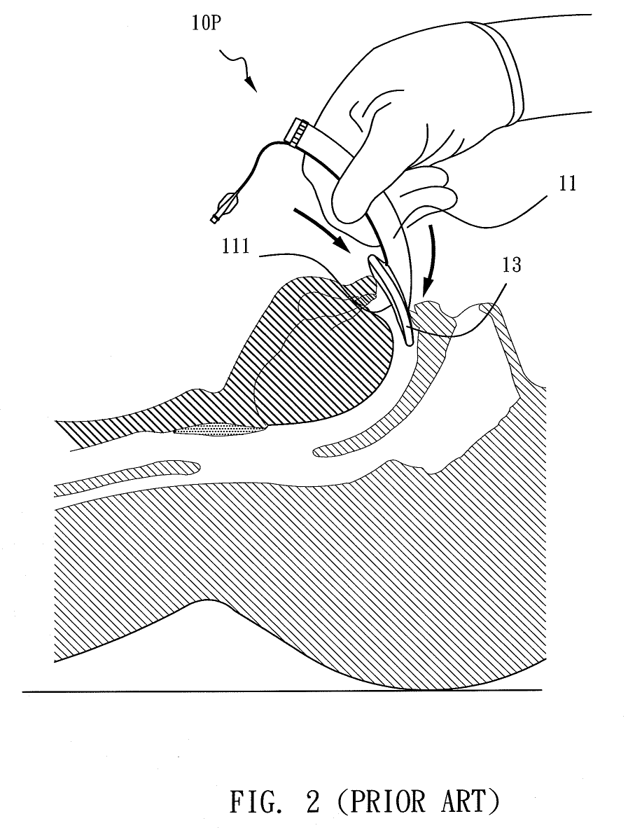

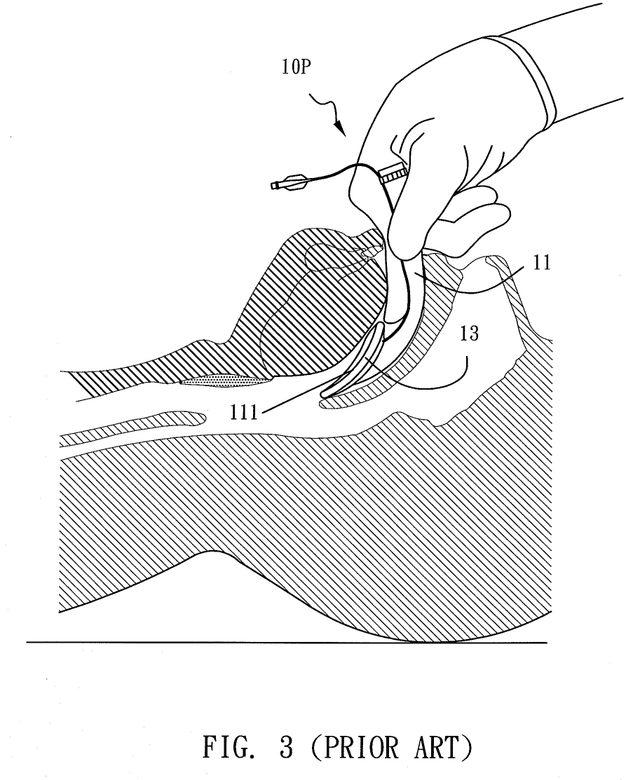

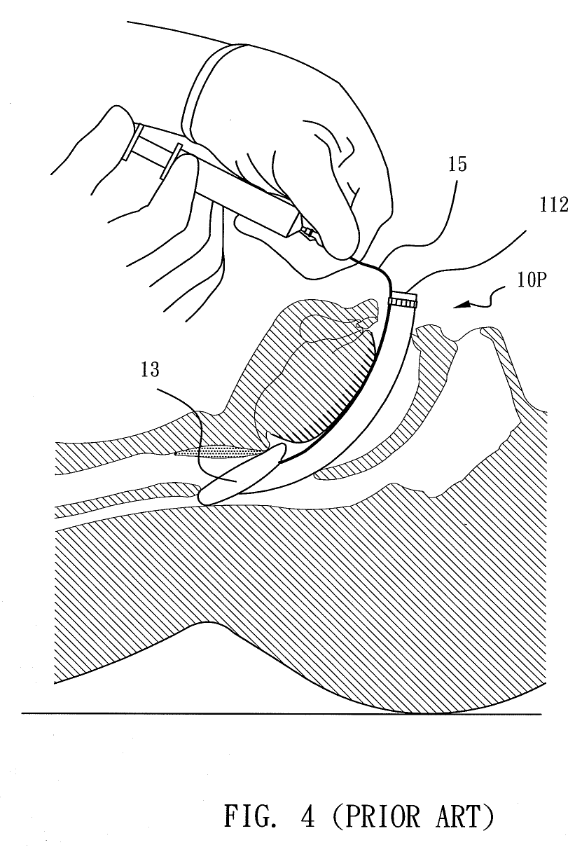

### DETAILED DESCRIPTION OF THE PREFERRED EMBODIMENT

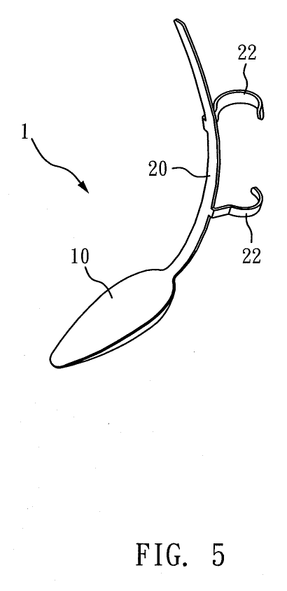

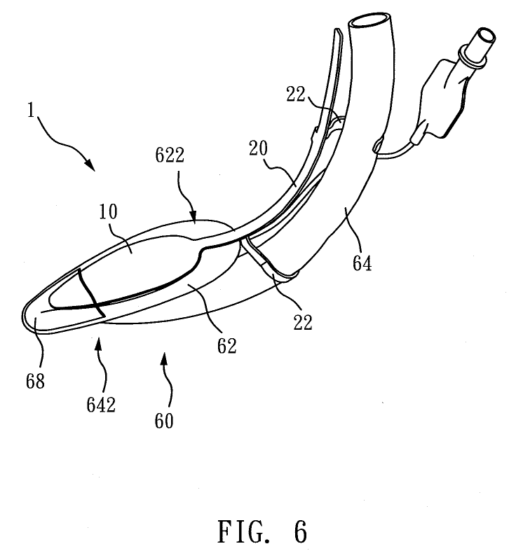

Please refer to both FIG. 5 and FIG. 6. FIG. 5 is an illustration of a first embodiment of a clip device of the present invention. FIG. 6 is an illustration of the first embodiment of the clip device installed on a laryngeal mask airway (LMA) of the present invention. The clip device 1 of the present invention comprises a main portion 10, a handle portion 20 and a mounting element 22. When the clip device 1 is connected to an LMA 60, the clip device 1 can strengthen the structure of the LMA 60, such that the LMA 60 can smoothly slide into a patient's body, and the patient's tongue can be lifted slightly by utilizing the main portion 10 of the clip device 1.

The LMA 60 comprises a gas filled portion 62 and a tube portion 64. The gas filled portion 62 is connected to one end 642 of the tube portion 64. The gas filled portion 62 is in the form of a circular hollow structure capable of storing gas therein. When the gas filled portion 62 is inflated with a predetermined volume of gas, the exterior of the gas filled portion 62 forms a soft surface. Since the LMA 60 is known as a prior art structure, there is no need for a further detailed description.

In this embodiment, the main portion 10 is in the form of a curved surface. In order to match the shape of the gas filled portion 62 of the LMA 60, the shape of the main portion 10 is egg-shaped (similar to the part of a spoon used for spooning liquids), such that the periphery of the main portion 10 can press the circular-shaped gas filled portion 62 (as shown in FIG. 6).

Please note that the main portion of the clip device of the present invention could be in the form of other shapes. For example, the main portion could be in the form of a plane, a curved surface or a frame (such as an O-shaped frame or a U-shaped frame). Furthermore, the shape of the main portion could be egg-shaped, oval-shaped or circular-shaped so as to match that of the gas filled portion 62.

The handle portion 20 is connected to the main portion 10. When the clip device 1 is connected to the LMA 60, the handle portion 20 is neighbor to the tube portion 64. More precisely, when a user (such as a doctor) operates the LMA 60 with the clip device 1 already connected thereto, he/she can hold both the handle portion 20 and the tube portion 64 at the same time. In this embodiment, the handle portion 20 has a flexible characteristic, such that it could associate with the tube portion 64, which is also flexible.

In this embodiment, in order to connect the handle portion 20 to the tube portion 64, the handle portion 20 comprises at least one mounting element 22. The mounting element 22 is used for connecting to the tube portion 64. The shape of each mounting element 22 matches that of the tube portion 64. In this embodiment, the at least one mounting element 22 comprises two C-shaped clip elements, wherein the internal diameter of each C-shaped clip element is equal to or slighter bigger than the external diameter of the tube portion 64. Further, there is a predetermined distance between the two C-shaped clip elements, and their C-shaped openings are facing opposite directions (as shown in FIG. 5). By means of the action of these two C-shaped clip elements, the handle portion 20 can easily connect to the tube portion 64 and can easily separate from the tube portion 64 as well.

Please note that the handle portion 20 is not always required to have the mounting element 22. The user could directly hold both the handle portion 20 and the tube portion 62 so as to prevent them from being separated.

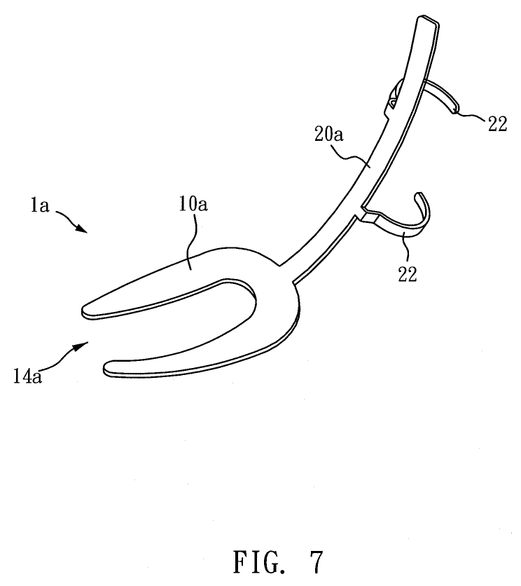

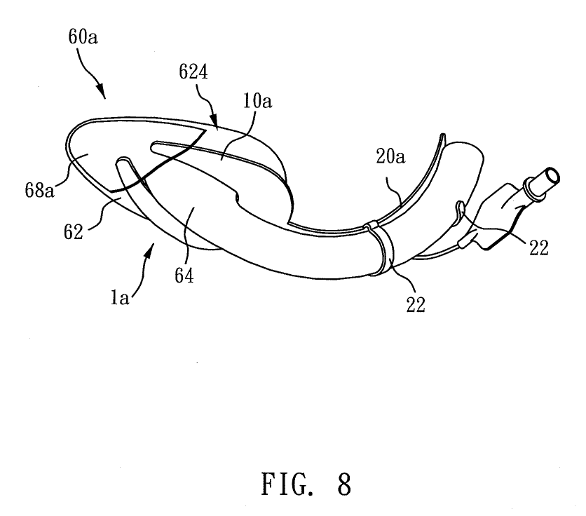

Please refer to FIG. 7 and FIG. 8. FIG. 7 is an illustration of a second embodiment of the clip device of the present invention. FIG. 8 is an illustration of the second embodiment of the clip device installed on the LMA of the present invention.

In this embodiment, the clip device 1a comprises a main portion 10a and a handle portion 20a. The main portion 10a can clip the tube portion 64 of the LMA 60. The main portion 10a is in a fork shape, and the fork forms an opening 14a. The opening 14a lies at the end of the clip device 1a, and the width of the opening 14a is equal to or slightly bigger than the external diameter of the tube portion 64, such that the tube portion 64 of the LMA 60 can be placed through the opening 14a for being mounted to the main portion 10a.

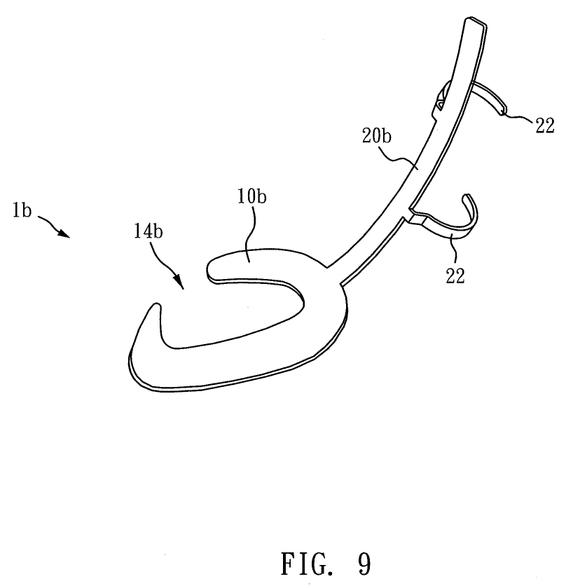

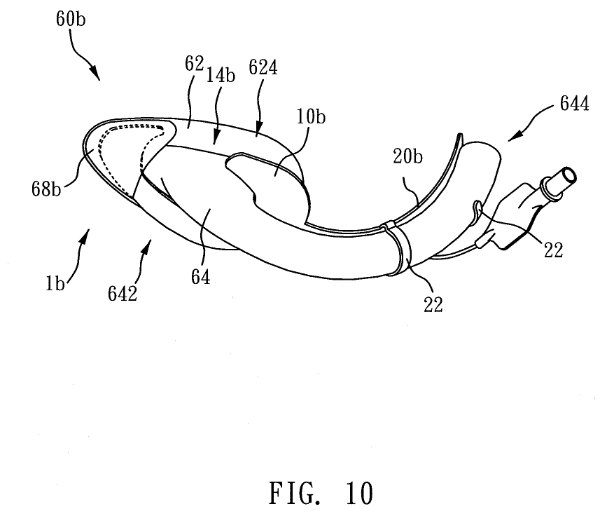

Next, please refer to FIG. 9 and FIG. 10. FIG. 9 is an illustration of a third embodiment of the clip device of the present invention. FIG. 10 is an illustration of the third embodiment of the clip device installed on the LMA of the present invention. In this embodiment, the clip device 1b comprises a main portion 10b and a handle portion 20b. The main portion 10b can clip the tube portion 64 of the LMA 60.

The difference between this embodiment and the second embodiment above is that the main portion 10b is C-shaped, and its opening 14b lies on one side of the main portion 10b, wherein the width of the opening 14b is equal to or slightly bigger than the external diameter of the tube portion 64, such that the tube portion 64 of the LMA 60 could be placed through the opening 14b for being mounted to the main portion 10b.

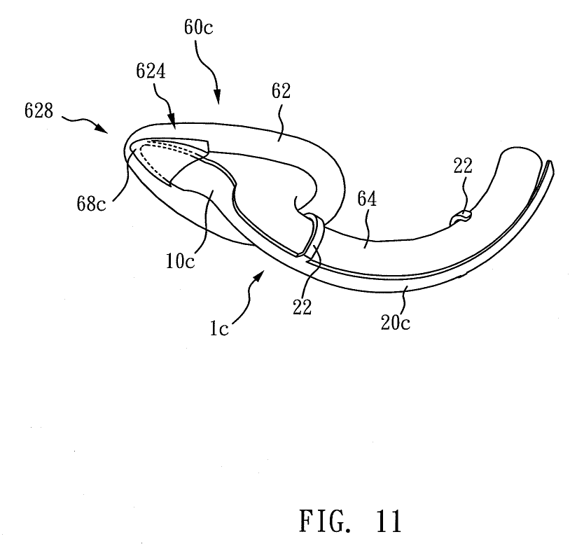

Please refer to FIG. 6, FIG. 8, FIG. 10 and FIG. 11 for explanations of the LMA of the present invention. The LMAs 60, 60a, 60b, 60c comprise a gas filled portion 62, a tube portion 64 and protection covers 68, 68a, 68b, 68c. The protection covers 68, 68a, 68b, 68c are connected to the gas filled portion 62, and the shapes and sizes of the protection covers 68, 68a, 68b, 68c are slightly different. The tube portion 64 comprises a first end 642 and a second end 644. The gas filled portion 62 is connected to the first end 642 of the tube portion 64. The gas filled portion 62 comprises an upper side 622 and a lower side 624. The protection cover 68 (as shown in FIG. 6) covers at least part of the upper side 622; while the protection covers 68a, 68b, 68c (respectively shown in FIG. 8, FIG. 10, FIG. 11) cover at least part of the lower side 624.

There are two purposes of setting the protection covers 68, 68a, 68b, 68c: one is to prevent the main portions 10, 10a, 10b, 10c from directly contacting the trachea and thus injuring the patient's tissue; the other is to improve the operation when the user uses the clip device 1b.

Please note that the position and number of the protection cover 68 is not limited to the above description. For example, there could be two protection covers, each respectively covering at least part of the upper side 622 and at least part of the lower side 624.

Because the front end of the gas filled portion 62 is the first part to contact the patient's trachea, the LMA 60, 60a, 60b, 60c would have better operational characteristics through the force applied by the user and the direction controlled by the user.

Please refer to FIG. 10. When the main portion 10b clips the tube portion 64 of the LMA 60a, the direction in which the tube portion 64 separates from the main portion 10b is different from the direction that is forced when using the clip device 1b. In addition, the tail end of the main portion 10b is placed in the protection cover 68b. Owing to the above double effects, the clip device 1b has a better mounting effect when clipping the LMA 60a.

Please note that, in this embodiment, the LMA could also be an LMA without the protection cover. At this time, the clip device 1b still has a fair mounting effect when clipping the LMA.

Please refer to FIG. 11 for an illustration of a fourth embodiment of the clip device installed on the LMA of the present invention. The LMA 60c comprises a gas filled portion 62, a tube portion 64, and a protection cover 68c. The clip device 1c comprises a main portion 10c and a handle portion 20c. In this embodiment, the shape of the clip device 1c matches that of the tube portion 64, such that the clip device 1c can press the tube portion 64. When the clip device 1c presses the tube portion 64, at least part of the main portion 10c is placed in the protection cover 68c.

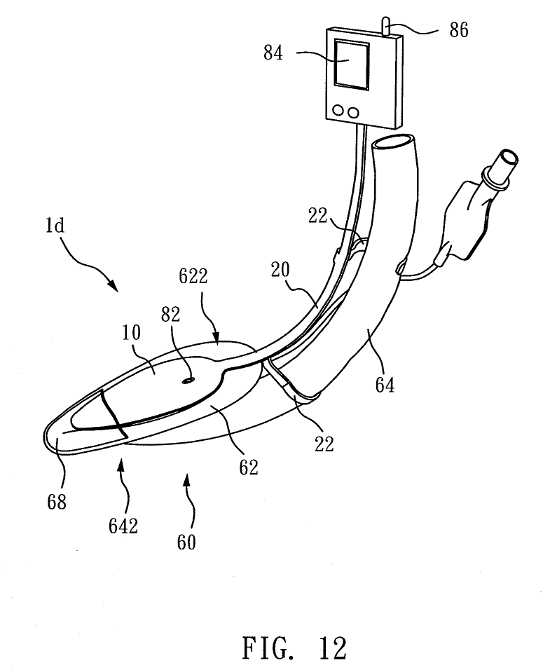

Please refer to FIG. 12 for an illustration of a fifth embodiment of the clip device installed on the LMA of the present invention. The major difference between this and the above embodiments is that the clip device 1 d comprises an image-capturing unit 82, a display unit 84 and a signal emission unit 86. The image-capturing unit 82 is used for capturing an image from a patient's upper airway. The captured image may then be directly shown on the display unit 84 connected to the handle portion 20, such that the user can determine the position of the LMA in the patient's upper airway. In addition, the image captured by the image-capturing unit 82, depending on the user's need, could be transmitted to an external display (not shown in figures) via the signal emission unit 86. Therefore, the clip device 1e of the present invention not only provides a function of facilitating the user to clip the LMA 60 but also allows the user to conveniently observe the installation status, so as to be provided with a more accurate status evaluation. Please note that the position of the image-capturing unit 82 is not limited to the above description.

Although the present invention has been explained in relation to its preferred embodiments, it is to be understood that many other possible modifications and variations can be made without departing from the spirit and scope of the invention as hereinafter claimed.

What is claimed is:

1. A clip device for clipping a laryngeal mask airway, the laryngeal mask airway comprising a gas filled portion and a tube portion, the clip device comprising:
a main portion for connecting to the laryngeal mask airway; and
a handle portion connected to the main portion, the handle portion being in long and thin shape.

2. The clip device as claimed in claim 1, wherein the main portion is in the form of a curved surface or a plane.

3. The clip device as claimed in claim 1, wherein the main portion is a frame.

4. The clip device as claimed in claim 3, wherein the shape of the main portion is egg-shaped, oval-shaped or circular-shaped.

5. The clip device as claimed in claim 3, wherein the main portion presses the gas filled portion of the laryngeal mask airway.

6. The clip device as claimed in claim 3, wherein the main portion is in a fork shape.

7. The clip device as claimed in claim 6, wherein the main portion clips the tube portion of the laryngeal mask airway.

8. The clip device as claimed in claim 6, wherein the main portion is C-shaped.

9. The clip device as claimed in claim 6, wherein the main portion is U-shaped.

10. The clip device as claimed in claim 1, wherein the handle portion further comprises at least one mounting element connected to the tube portion.

11. The clip device as claimed in claim 10, wherein each mounting element is a C-shaped clip element.

12. A laryngeal mask airway capable of connecting to a clip device, the clip device comprising a main portion, the laryngeal mask airway comprising:
a tube portion comprising a first end and a second end;
a gas filled portion connected to the first end of the tube portion, the gas filled portion comprising an upper side and a lower side; and
at least one protection cover connected to the gas filled portion, the at least one protection cover used for holding at least part of the main portion.

13. The laryngeal mask airway as claimed in claim 12, wherein the at least one protection cover covers at least part of the upper side.

14. The laryngeal mask airway as claimed in claim 12, wherein the at least one protection cover covers at least part of the lower side.

15. The laryngeal mask airway as claimed in claim 12, wherein the number of the at least one protection cover is two.

16. The laryngeal mask airway as claimed in claim 15, wherein the at least one protection cover respectively covers at least part of the upper side and at least part of the lower side.
